(19) United States
(12) Patent Application Publication
(10) Pub. No.: US 2007/0100270 A1
(43) Pub. Date: May 3, 2007

(54) Navel concealer

(76) Inventor: Sheryl L. Parente, Bryn Mawr, PA (US)

Correspondence Address: LAW OFFICE OF STEVEN B. LEAVITT, L.L.P., P.O. BOX 537, ROWLETT, TX, 75030-0537, US

(21) Appl. No.: 11/263,477

(22) Filed: Oct. 31, 2005

(51) Int. Cl. A61F 15/00

(52) U.S. Cl. 602/52

(57) ABSTRACT

The present invention is directed to a concealment device for a protruding navel. The concealment device includes a sheet having a top surface with a relatively flat profile and a bottom surface. The sheet is comprised of flexible material, and an adhesive is disposed on the bottom surface of the sheet. The bottom surface of the sheet is capable of being adhered to an abdomen such that a protruding navel is concealed, thereby creating the appearance of a smooth abdomen.

### BACKGROUND OF THE INVENTION

1. Technical Field

This invention relates to a concealment device for a bodily protrusion, and more particularly, to a concealment and protective device for a protruding navel.

2. Description of Related Art

During pregnancy, as the baby grows inside the womb, the abdomen expands which causes the navel or belly button to become stretched. For the majority of pregnant women, the navel first becomes flattened and eventually protrudes from the abdomen as the pregnancy progresses. This condition even occurs in women who typically have deep or “innie” navels prior to pregnancy.

The navel may begin protruding from the abdomen as early as twelve to fourteen weeks into the pregnancy and remains in this protruding state until the woman gives birth and sometimes for a few weeks afterwards. While a protruding belly button is not unhealthy, for some women, the protruding belly button may become sensitive to touch, sore or even painful. In addition, the majority of pregnant women are very self conscious of the way their expanded navel or “outie” looks under clothing.

Also, as many as 10% of the United States population has an “outie” or protruding navel. Most Doctors believe the reasons for a protruding navel is purely random and is caused because the belly button—which actually is a scar—simply grows as it wants, and one time out of 10, it grows out. Often, those with a protruding navel feel like an outcast and some seek plastic surgery to remove the protruding navel. Therefore, what is needed is a way to protect and conceal an “outie” or protruding navel.

One method to conceal a protruding navel is with tape. However, tape is not breathable and can cause irritation of the skin. Others have tried to cover a protruding navel with a bandage. However, the padded area of the bandage strip, which is often rectangular and bulkier than any other part of the bandage, exhibits a tendency to lift in response to the protruding navel and the lifting creates a tenting effect of the bandage strip. Even when applied properly, a bandage will exhibit a propensity to allow the protruding navel to erect beneath the gauze area. The surrounding adhesive thus remains intact with the surrounding tissue, but the adhesive-free gauze area lifts off of the skin. The result is a relatively pointed “peak” area where the protruding navel is.

The profile of the padded portion of the bandage strip is, therefore, visible through clothes, especially sheer and lightweight fabrics. Such a heightened profile is undesirable and produces an unnatural look and rather than concealing the protruding navel, the heightened profile of a bandage strip, actually draws additional attention to the area, thereby exacerbating the problem, rather than solving it.

In addition, in most bandages, the adhesive is not fully around the area covering the navel and often the bandage is difficult to secure and will not remain in the exact location it was positioned. Also, the outside surface of bandages will often catch on the cloths of the user and draw even more attention to the navel region. What is needed is a smooth seamless bully button concealer that does not gap and is stretchable. The navel concealer should produce a smooth profile when worn and should not draw unwanted attention to the navel region. It would be beneficial if the navel concealer could protect the sore or sensitive navel of a pregnant women.

### SUMMARY OF INVENTION

The present invention solves the above-described problem by providing a device that conceals a protruding navel to provide a smooth abdomen appearance. The concealment device acts to cover the navel while gently pressing the protruding navel back into its original non-protruding position or at least provides a visually flat surface or the appearance of a flat surface instead of a visible protruding navel.

The concealment device of the present invention includes a sheet comprised of flexible material having a top surface and a bottom surface. An adhesive is disposed on the bottom surface of the sheet such that the bottom surface is capable of being adhered to an abdomen so that a protruding navel is concealed, thereby creating the appearance of a smooth abdomen.

More particularly, the adhesive may be a pressure sensitive adhesive, and is preferably located along the perimeter of the bottom surface of the sheet. In addition, the device may include at least one removable release cover secured to the bottom surface of the sheet. The release cover protects the bottom surface and adhesive prior to use of the concealment device.

Another aspect of the present invention is directed to a device for concealing a protruding navel comprising a sheet having a top surface and a bottom surface. The sheet is comprised of flexible material, and a protective area is located on the bottom surface of the sheet. In addition, pressure sensitive adhesive is disposed along the perimeter of the bottom surface of the sheet. The sheet is capable of being adhered to an abdomen such that a protruding navel is concealed, thereby creating the appearance of a smooth abdomen.

Other features and advantages of the present invention will become apparent upon reading the following detailed description of embodiments of the invention, when taken in conjunction with the appended claims.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein

FIG. 1 is a top view of a concealment device made in accordance with an embodiment of the present invention;

FIG. 2 is a bottom view of a concealment device made in accordance with an embodiment of the present invention;

FIG. 3 is a bottom view of a concealment device made in accordance with an alternative embodiment of the present invention;

FIG. 4 is a top view of a concealment device made in accordance with an alternative embodiment of the present invention;

FIG. 5 is a bottom view of a concealment device made in accordance with an alternative embodiment of the present invention; and

FIG. 6 is a cross-sectional view of a concealment device made in accordance with an alternative embodiment of the present invention.

### DETAILED DESCRIPTION OF THE INVENTION

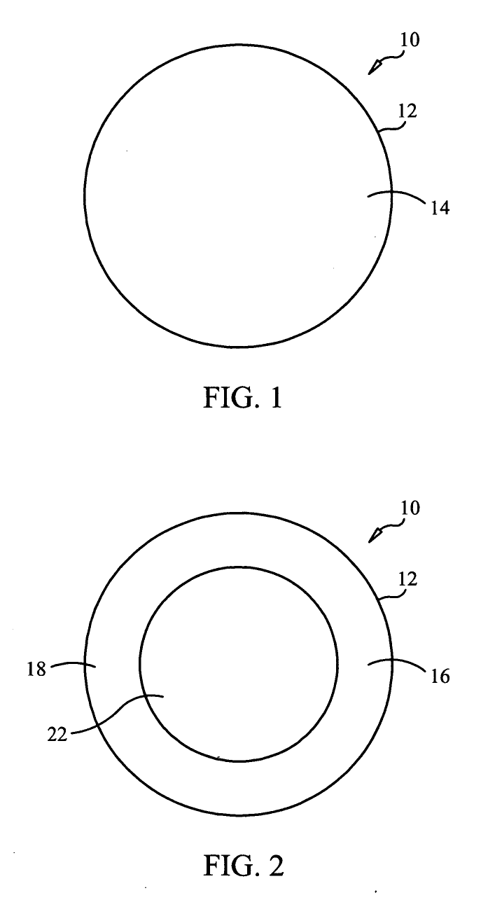

Referring to FIGS. 1 and 2, shown is concealment device 19 for concealing a protruding navel. Concealment device 10 includes sheet 12 having top surface 14 and bottom surface 16. Sheet 12 may be comprised of any breathable and flexible material known to those having skill in the art, which is capable of conforming to the contours of an abdomen to create a smooth abdomen appearance. Suitable material for sheet 12 may include, but is limited to, fabric, plastic, paper, acrylic, cotton, linen, nylon, woven cloth like material, non-woven cloth like material, satin, sateen, micro-fiber polyester, micro-fiber nylon, plastic mesh, metal mesh, or other similar material that can breath and is flexible. The material should be breathable so as not to trap moisture. In addition, the material should be flexible so as to follow the abdomen's contours and remain in contact with the abdomen when the body moves. Furthermore, the flexible material is preferably a sheer material which prevents concealment device 10 from being seen through clothing worn by the user. Top surface 14 of concealment device 10 is smooth so that clothing worn over the device has freedom of movement and will not snag or get caught on the device.

Adhesive 18 is disposed on bottom surface 16 of sheet 12. Adhesive 18 may be any adhesive known to those having skill in the art, that is suitable for use on human skin, and preferably for use on sensitive human skin. In addition, adhesive 18 should be strong enough to maintain concealment device 10 on an abdomen for a period of approximately 12 hours, yet allow for removal of the concealment device from the skin without injuring the skin; use of such adhesive is common in the art. Preferably, adhesive 18 is a pressure sensitive adhesive.

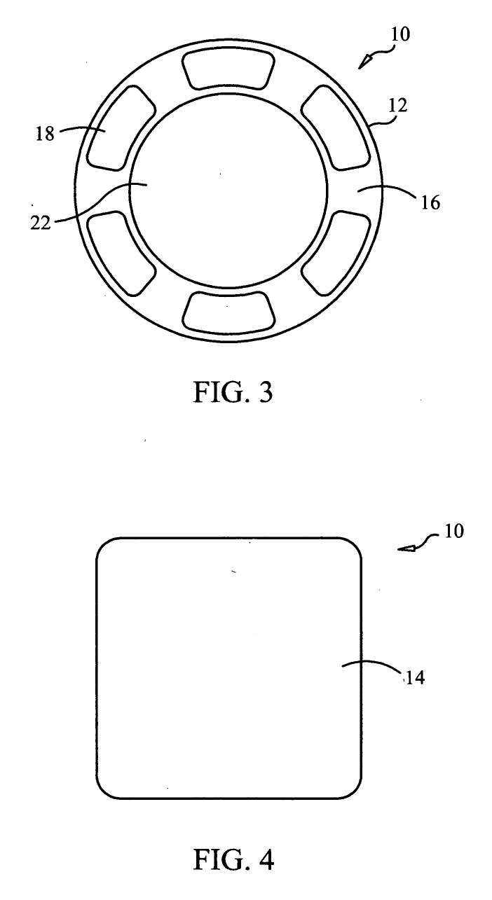

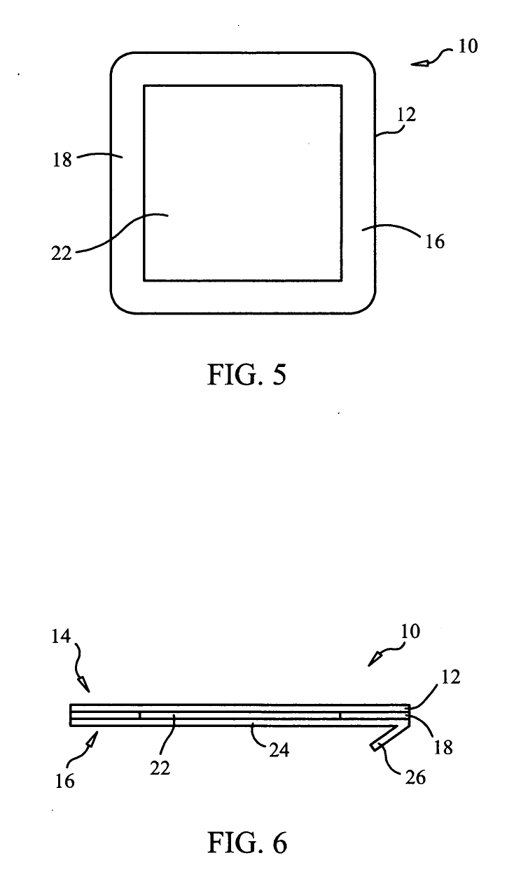

As illustrated in FIGS. 2 and 5, adhesive 18 is located along at least a portion of the perimeter of sheet bottom surface 16. Adhesive 18 may cover a continuous area along the perimeter of bottom surface 16, as shown in FIGS. 2 and 5 or alternatively, adhesive 18 may be arranged in a discontinuous plurality of deposits along the perimeter of bottom surface 16, as illustrated in FIG. 3. Also, the adhesive ay be applied in a discontinuous pattern such as dot-distribution, cross hatching, or any other pattern.

Concealment device 10 may be any shape which provides for effective concealment of a protruding navel, and creates the appearance of a smooth abdomen. The profile of concealment device 10 may be circular, triangular, square, rectangular, or any other polygon. In a preferred embodiment, concealment device 10 is circular (FIGS. 1-3) or square (FIGS. 4 and 5) in shape. A square shaped concealment device 10, preferably has rounded corners, as illustrated in FIGS. 4 and 5. In addition, concealment device 10 may be in a novelty shape such as a heart, baby, baby bottle, stroller, pacifier, bear or some other design. Preferably the design has a baby theme.

Concealment device 10 preferably has a length and width between about 1.0 inch to about 12.0 inches. More preferably, concealment device 10 has a length and width between about 2.0 inches to about 6.0 inches. If concealment device 10 is circular in shape, the device preferably has a diameter between about 1.0 inch to about 12.0 inches. More preferably, the device 10 has a diameter between about 2.0 inches to about 4.0 inches.

In addition, concealment device 10 may be produced in a variety of colors so the device will blend with the skin color or shade of clothing worn by the user. For example, if the user is wearing a blue shirt, then sheet 12 or top surface 12 may be a similarly colored blue. The colors may include, but are not limited to yellow, pink, red, blue, green, orange, purple, brown, black, white, or flesh color.

Protective area 22 may be located on bottom surface 16 of sheet 12. Protective area 22 is preferably located in the center of bottom surface 16, and covers and protects the navel when concealment device 10 is adhered to the abdomen. Protective area 22 is particularly useful in guarding a sensitive protruding navel against contact with clothing or other objects which may cause irritation. Protective area 22 is preferably a soft breathable material such as gauze, cotton or some other similar material.

As shown in FIG. 6, concealment device 10 of the present invention preferably includes at least one removable release cover 24 secured to bottom surface 16 of sheet 12. Release cover 24 protects bottom surface 16 of sheet 12, including adhesive 18 and protective area 22, prior to use of concealment device 10. Release cover 24 is conventional, and may include a silicone-coated substrate permitting quick release or removal of the release cover 24 from adhesive 18. In addition, release cover 24 may include flap 26 to be grasped for removal of the release cover 24.

Top surface 14 has a relatively flat profile unlike bandages and the flat profile prevents concealment device 10 from producing the heightened profile of a bandage strip and drawing attention to the area, exacerbating the problem, rather than solving it.

It should be understood that the foregoing relates to exemplary embodiments of the invention and that modifications may be made without departing from the spirit and scope of the invention as set forth in the following claims.

What is claimed is:

1. A device for concealing a protruding navel comprising:
a sheet comprised of flexible material, the sheet having a top surface with a relatively flat profile and a bottom surface; and
a pressure sensitive adhesive disposed on the bottom surface wherein the bottom surface of the sheet is capable of being adhered to an abdomen such that a protruding navel is concealed.

2. The device of claim 1, wherein the adhesive is located along the perimeter of the bottom surface.

3. The device of claim 1, wherein the sheet is circular, square, rectangular, oval, or triangular in shape.

4. The device of claim 1, wherein the sheet is in the shape of a heart, bottle, baby, bear, stroller or pacifier.

5. The device of claim 1, wherein the flexible material has a smooth surface so as not to restrict the flow of fabric over it.

6. The device of claim 1, wherein the flexible material is comprised of plastic, acrylic, sateen, satin, micro fiber polyester, micro fiber nylon, gauze, cotton, plastic mesh or metal mesh.

7. The device of claim 1, wherein the flexible material is breathable.

8. The device of claim 1 wherein the flexible material is sheer.

9. The device of claim 1, wherein the sheet has a length and width between about 2 and about 12 inches.

10. The device of claim 1, wherein the sheet has a diameter between about 1 and about 12 inches.

11. The device of claim 1, further comprising at least one removable release cover secured to the bottom surface wherein the release cover protects the bottom surface and adhesive prior to use of the device.

12. The device of claim 1, further comprising a protective area located on the bottom surface of the sheet, the protective area covers the navel when the device is adhered to the abdomen.

13. The device of claim 10, wherein the protective area is comprised of gauze or cotton.

14. A device for concealing a protruding navel comprising:
a sheet comprised of flexible breathable material, the sheet having a top surface with a relatively flat profile and a bottom surface;
a protective area located on the bottom surface of the sheet; and
pressure sensitive adhesive disposed along the perimeter of the bottom surface;
the bottom surface of the sheet is capable of being adhered to an abdomen such that a protruding navel is concealed.

15. The device of claim 14, wherein the sheet is circular, square, rectangular, oval, or triangular in shape.

16. The device of claim 14, wherein the sheet is in the shape of a heart, bottle, baby, bear, stroller or pacifier.

17. The device of claim 14, wherein the flexible material has a smooth surface so as not to restrict the flow of fabric.

18. The device of claim 14, wherein the sheet has a length and width between approximately 2 and approximately 12 inches.

19. The device of claim 14, wherein the sheet has a diameter between about 1 and about 12 inches.

20. The device of claim 14, further comprising at least one removable release cover secured to the bottom surface, the release cover protects the bottom surface and adhesive prior to use of the device.
